(19) United States
(12) Patent Application Publication
(10) Pub. No.: US 2005/0234103 A1
(43) Pub. Date: Oct. 20, 2005

(54) Crystalline forms of rabeprazole sodium

(76) Inventors: Manne Satyanarayana Reddy, Hyderabad (IN); Sajja Eswaraiah, Hyderabad (IN); Vijava Bhaskar Bolugoddu, Hyderabad (IN); Ramchandra Reddy Pingili, Hyderabad (IN); Madhusudhan Reddy Ganta, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/505,826

(22) PCT Filed: Mar. 25, 2003
(86) PCT No.: PCT/US03/09307

§ 371 Date: Jun. 13, 2005

(30) Foreign Application Priority Data
Mar. 26, 2002 (IN) 207/MAS/02

(51) Int. Cl. A61K 31/4439; C07D 43/02

(52) U.S. Cl. 514/338; 546/273.7

(57) ABSTRACT

The present invention relates to novel polymorphic forms of Rabeprazole sodium. The present invention also relates to methods of making polymorphic forms of Rabeprazole sodium. Achiphex7 (Rabeprazole sodium) is an inhibitor of the gastric proton pump. It causes dose-dependant inhibition of acid secretion and is useful as an antiulcer agent. The chemical designation of Rabeprazole sodium is 2-[[[4-(3-methoxypropoxy)-3 methyl-2-pyridinyl]-methyl]sulfinyl]-1H-benzimidazole sodium. It may be represented by Formula (1).

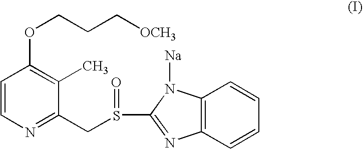

The present invention relates to novel polymorphic forms of Rabeprazole sodium. The present invention also relates to methods of making polymorphic forms of Rabeprazole sodium.

Achiphex7 (Rabeprazole sodium) is an inhibitor of the gastric proton pump. It causes dose-dependant inhibition of acid secretion and is useful as an antiulcer agent. The chemical designation of Rabeprazole sodium is 2-[[[4-(3-methoxypropoxy)-3-methyl-2-pyridinyl]-methyl]sulfinyl]-1H-benzimidazole sodium. It may be represented by Formula (1):

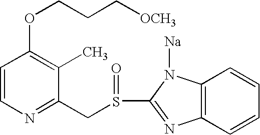

U.S. Pat. No. 5,045,552 incorporated herein by reference describes the synthesis of Rabeprazole and its sodium salt. Rabeprazole is prepared by oxidizing 2-[{4-(3-methoxypropoxy)-3-methylpyridine-2-yl}methylthio]-1H-benzimidazole with m-chloroperbenzoic acid to afford the Rabeprazole base, which is then converted to its sodium salt by aqueous sodium hydroxide solution.

Polymorphism is the occurrence of different crystalline forms of a single compound and it is a property of some compounds and complexes. Thus, polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-Ray diffraction peaks. Since the solubility of each polymorph may vary, identifying the existence of pharmaceutical polymorphs is essential for providing pharmaceuticals with predictable solubility profiles. It is desirable to investigate all solid forms of a drug, including all polymorphic forms, and to determine the stability, dissolution and flow properties of each polymorphic form. Polymorphic forms of a compound can be distinguished in the laboratory by X-Ray diffraction spectroscopy and by other methods such as, infrared spectrometry.

Crystal forms of Rabeprazole are mentioned in Japanese Patent 2001-39975 and they are designated as crystal I and II. However crystal I is not identified by recognized methods of crystal structure identification such as X-Ray diffraction.

The crystal II of Rabeprazole sodium, however, is discussed in detail and is characterized by its X-Ray diffraction spectroscopy, Infrared spectrometry and Differential Scanning Colorimetry.

The process for the preparation of crystal II as disclosed in the Japanese Patent specification comprises crystallization of amorphous Rabeprazole sodium or acetone complex of Rabeprazole sodium in one or more solvents selected from ethyl acetate, isopropyl acetate, isobutyl acetate, ethyl propionate, isobutyl propionate and ethyl butyrate.

The X-ray diffractogram for crystal II as in Japanese Patent 2001-39975 is as follows:

| 2 theta(°) | I/Io(%) |
|---|---|
| 19.52 | 100 |
| 17.20 | 41 |
| 26.60 | 28 |
| 20.92 | 18 |
| 18.04 | 17 |
| 24.76 | 13 |
| 21.20 | 12 |
| 14.22 | 10 |
| 17.60 | 10 |
| 25.00 | 10 |
| 29.40 | 10 |
| 28.76 | 9 |
| 27.56 | 7 |
| 27.76 | 7 |
| 12.54 | 5 |
| 13.20 | 5 |
| 24.38 | 5 |
| 28.50 | 5 |
| 34.04 | 5 |
| 13.80 | 4 |
| 22.64 | 4 |
| 24.16 | 4 |
| 30.00 | 4 |
| 31.62 | 3 |
| 12.82 | 3 |
| 34.92 | 2 |
| 25.92 | 2 |
| 11.84 | 2 |
| 8.88 | 1 |
| 9.64 | 1 |

The present invention provides for novel crystalline forms of Rabeprazole sodium, which are designated as Form X and Form Y for convenience. Another aspect of the invention is to provide hydrates of Form X and Form Y of Rabeprazole.

Another aspect of the present invention is to provide processes for the preparation of the crystalline Form X and Form Y of Rabeprazole sodium. The processes are commercially viable and well-suited for industrial scale up.

### BRIEF DESCRIPTION OF ACCOMPANYING DRAWINGS

FIG. 1 is characteristic X ray powder diffractogram of Form X of Rabeprazole sodium.

FIG. 2 is Differential Scanning Calorimetry thermogram of Form X of Rabeprazole sodium.

FIG. 3 is characteristic X ray powder diffractogram of Form Y of Rabeprazole sodium.

FIG. 4 is Differential Scanning Calorimetry thermogram of Form Y of Rabeprazole sodium.

The present invention provides novel crystalline Form X and Form Y of Rabeprazole sodium. Each of these solid-state forms includes non-solvated and hydrated crystalline forms. The crystalline Form X and Form Y of the present invention may be characterized by their X Ray powder diffraction patterns. The X-Ray diffraction patterns of Form X and Y of Rabeprazole sodium were measured on a Bruker Axs, D8 Advance Powder X-ray Diffractometer with Cu K alpha-1 Radiation source.
Scan range=3-45° (2 Theta)

Scan speed=step size 0.02°, time for step is 0.4 seconds.

| | |
|---|---|
| Sampling time | Scan time: 14:0.40 min |
| Scan mode | Continuous |
| Reflection | Geometry is Reflection (Not transmission) |
| Scan type | Locked coupled |

Voltage=45 KV,
Current=35 mA

Crystalline Form X has X-ray powder diffraction pattern essentially as shown in the Table 1. The X-ray powder diffraction pattern is expressed in term of the 2θ, and relative intensities (cps).

TABLE 1

| 2θ(°) | Intensity (cps) |
|---|---|
| 5.13 | 1184 |
| 6.606 | 225 |
| 20.01 | 215 |
| 23.469 | 193 |
| 8.569 | 169 |
| 12.923 | 154 |
| 20.539 | 135 |
| 22.177 | 131 |
| 24.81 | 125 |
| 10.565 | 125 |
| 12.161 | 116 |
| 9.353 | 113 |
| 18.173 | 99.3 |
| 17.309 | 85.3 |
| 14.864 | 81.3 |
| 25.494 | 75.3 |
| 16.372 | 69.9 |
| 14.414 | 60.2 |
| 7.244 | 57.5 |
| 19.072 | 56.2 |

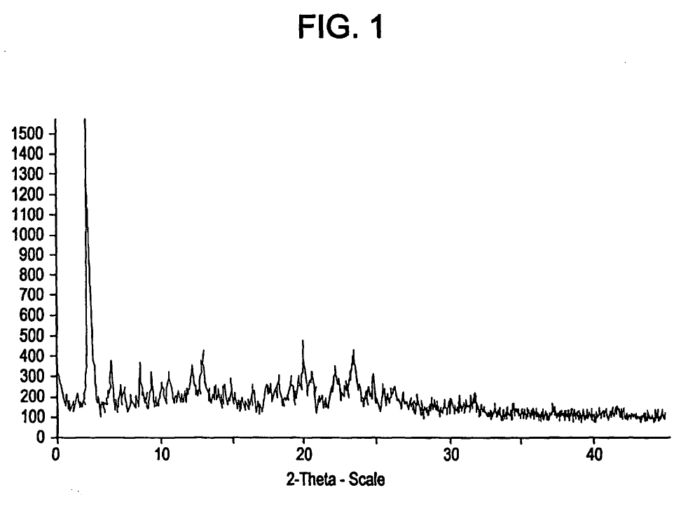

The present invention also provides Form X of Rabeprazole sodium that is characterized by its X Ray powder diffraction substantially as depicted in FIG. 1.

The present invention also provides the Differential Scanning Calorimetry thermogram of Form X of Rabeprazole sodium. The Differential Scanning Calorimetry thermogram exhibits a significant endo-exo pattern at 154.62° C. and 214.65° C.

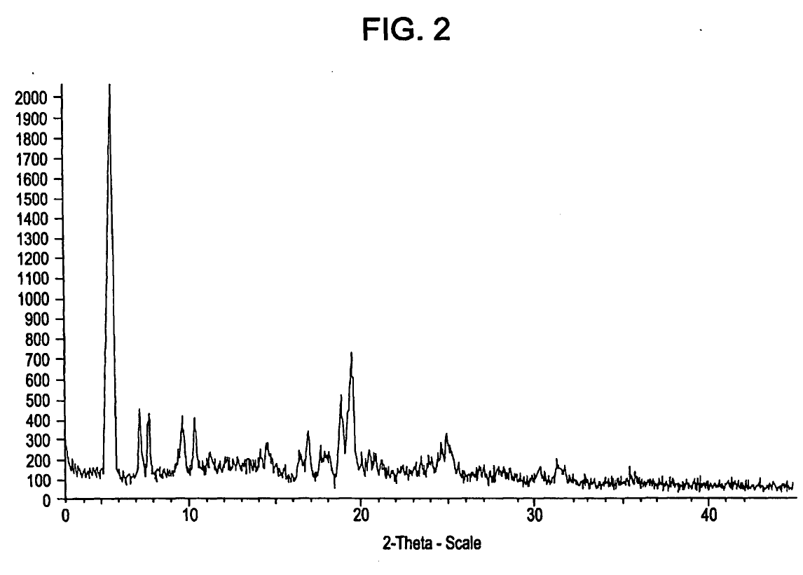

The present invention also provides Differential Scanning Calorimetry thermogram of Form X of Rabeprazole sodium substantially as depicted in FIG. 2.

The present invention also provides melting range (capillary method) of crystalline Form X at 140-150° C.

Accordingly the present invention also provides a process for the preparation of Form X of Rabeprazole sodium, which comprises the steps of:
a) dissolving 2-[[[4-(3-methoxypropoxy)-3-methyl-2-pyridinyl]-methyl]sulfinyl]-1H-benzimidazole in a solvent comprising C₁-C₄ alkanol solution of a sodium hydroxide or a mixture thereof, followed by distillation off of the solvent;
b) adding a chlorinated C₁-C₃ hydrocarbon solvent or a mixture;
c) distilling the residual C₁-C₄ alkanol of a sodium hydroxide azetropically under reduced pressure from the reaction solution of b);
d) adding a chlorinated C₁-C₃ hydrocarbon solvent and an C₅-C₁₀ alkane or a C₅-C₁₀ cyclic alkane or mixtures thereof; accompanied by stirring; and isolating Form X of Rabeprazole sodium.

The 2-[[[4-(3-methoxypropoxy)-3-methyl-2-pyridinyl]-methyl]sulfinyl]-1H-benzimidazole may be prepared by any process. Preferably, the 2-[[[4-(3-methoxypropoxy)-3-methyl-2-pyridinyl]-methyl]sulfinyl]-1H-benzimidazole is prepared as in the Reference Example.

The Form X of Rabeprazole sodium may be isolated using methods such as filtratipn or centrifugation.

The C₁-C₄ alkanol of a sodium hydroxide may be selected from methanolic sodium hydroxide, ethanolic sodium hydroxide or isopropanolic sodium hydroxide or mixtures thereof, preferably, methanolic sodium hydroxide.

The C₁-C₄ alkanol of a sodium hydroxide may be prepared by mixing the C₁-C₄ alkanol with sodium hydroxide in situ.

The chlorinated C₁-C₃ hydrocarbon solvent may be selected from dichloromethane, dichloroethane, trichloroethane, tetrachloroethane, dichloropropane, dichloropropane, chloroform or carbon tetrachloride, preferably dichloromethane.

The alkane may be selected from pentane, hexane, heptane, petroleum ether, octane, i-octane, nonane, or decane or mixtures thereof, preferably petroleum ether.

The cyclic alkane may be selected from cyclopentane, cyclohexane, or cycloheptane or mixtures thereof, preferably cyclohexane.

In step a) the preferred ratio of 2-[[[4-(3-methoxypropoxy)-3-methyl-2-pyridinyl]-methyl]sulfinyl]-1H-benzimidazole to the C₁-C₄ alkanol of a sodium hydroxide is 1:2 w/v (i.e. 1 gram of 2-[[[4-(3-methoxypropoxy)-3-methyl-2-pyridinyl]-methyl]sulfinyl]-1H-benzimidazole to 2 ml of the C₁-C₄ alkanol of a sodium hydroxide).

In step b) the ratio of residual mass of step a) to chlorinated C₁-C₃ hydrocarbon is 1:1-10 w/v, preferably 1:2 w/v.

In step d) the ratio of chlorinated hydrocarbon solvent to alkane and/or cyclic alkane is 1:5-15 v/v; preferably 1:5-10 v/v and more preferably 1:5 v/v.

Another aspect of this invention is to provide crystalline Form Y of Rabeprazole. Form Y of Rabeprazole has X-ray powder diffraction pattern essentially as shown in the Table 2. The X-ray powder diffraction pattern is expressed in terms of the 2θ, the relative intensities (cps).

TABLE 2

| 2θ(°) | Intensity (cps) |
|---|---|
| 5.61 | 1635 |
| 19.442 | 546 |
| 18.816 | 329 |
| 7.725 | 285 |
| 7.207 | 242 |
| 9.649 | 235 |
| 10.352 | 219 |
| 16.899 | 186 |
| 24.943 | 162 |
| 16.418 | 104 |
| 14.546 | 103 |
| 11.231 | 77.0 |

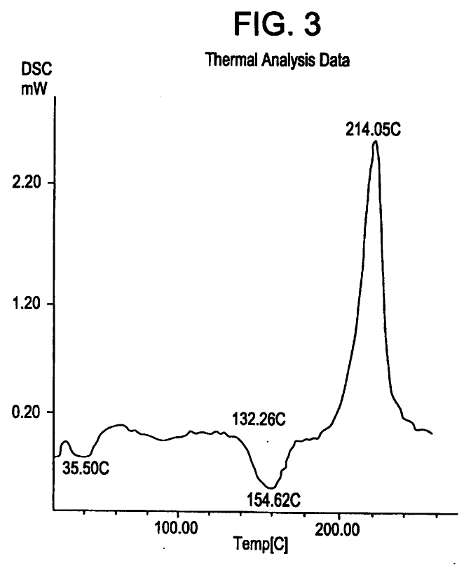

The present invention also provides Form Y of Rabeprazole sodium that is characterized by its X Ray powder diffraction substantially as depicted in FIG. 3.

The present invention also provides the Differential Scanning Calorimetry thermogram of Form Y of Rabeprazole sodium. The Differential Scanning Calorimetry thermogram exhibits a significant endo-exo pattern at 182.61° C. and 215.57° C.

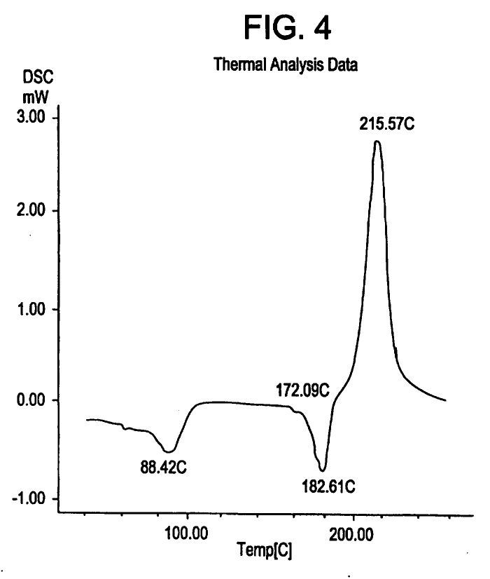

The present invention also provides Differential Scanning Calorimetry thermogram of Form Y of Rabeprazole substantially as depicted in FIG. 4.

Accordingly the present invention also provides a process for the preparation of Form Y of Rabeprazole sodium, which comprises the steps of:
a) dissolving 2-[[[4-(3-methoxypropoxy)-3-methyl-2-pyridinyl]-methyl]sulfinyl]-1H-benzimidazole in a solvent comprising C₁-C₄ alkanol of sodium hydroxide or mixtures thereof, followed by distilling the solvent from the reaction solution;
b) optionally adding chlorinated a C₁-C₃ hydrocarbon solvent to the residual mass obtained in step a);
c) distilling the residual C₁-C₄ alkanol of sodium hydroxide azeotropically under reduced pressure from the reaction solution of step b);
d) adding to the residue obtained in step c) either a C₃-C₅ straight or branched chain alcohol and an ether solvent having a C₁-C₄ straight or branched carbon chain; and isolating Form Y of Rabeprazole sodium.

The 2-[[[4-(3-methoxypropoxy)-3-methyl-2-pyridinyl]-methyl]sulfinyl]-1H-benzimidazole may be prepared by any process. Preferably, the 2-[[[4-(3-methoxypropoxy)-3-methyl-2-pyridinyl]-methyl]sulfinyl]-1H-benzimidazole is prepared as in the Reference Example.

The Form Y of Rabeprazole sodium may be isolated using methods such as filtration or centrifugation.

The C₁-C₄ alkanol of a sodium hydroxide may be selected from methanolic sodium hydroxide, ethanolic sodium hydroxide or isopropanolic sodium hydroxide or mixtures thereof, preferably, methanolic sodium hydroxide.

The C₁-C₄ alkanol of a sodium hydroxide may be prepared by mixing the C₁-C₄ alkanol with sodium hydroxide in situ.

The chlorinated C₁-C₃ hydrocarbon solvent may be selected from dichloromethane, dichloroethane, trichloroethane, dichloropropane, tetrachloroethane, dichloropropane, chloroform or carbon tetrachloride, preferably dichloromethane.

The C₃-C₅ straight or branched chain alcohol may be selected from n-propanol, n-butanol, 2-butanol, or tert. Butanol, preferably n-butanol.

The ether solvent having a C₁-C₄ straight or branched carbon chain may be selected from diethyl ether, methyl ethyl ether, diisobutyl ether, ditertiary Butyl ether or tertiary Butyl methyl ether.

In step a) the preferred ratio of 2-[[[4-(3-methoxypropoxy)-3-methyl-2-pyridinyl]-methyl]sulfinyl]-1H-benzimidazole to the C₁-C₄ alkanol of a sodium hydroxide is 1:2 w/v (i.e. 1 gram of 2-[[[4-(3-methoxypropoxy)-3-methyl-2-pyridinyl]-methyl]sulfinyl]-1H-benzimidazole to 2 ml of the C₁-C₄ alkanol of a sodium hydroxide.

In step b) the ratio of residual mass to chlorinated C₁-C₃ hydrocarbon is 1:1-10 w/v, preferably 1:2 w/v.

In step d) the ratio of alcohol to ether solvent is 1:10-20 v/v, preferably 1:15-20 v/v, more preferably 1:16 v/v.

2-[(4-3-methoxypropoxy)-3-methylpyridine-2-yl}methylthio]-1H-benzimidazole (I) can be prepared using the methods disclosed in the U.S. Pat. No. 5,045,552.

The oxidation step of (I) maybe carried out in solvents such as C₁-C₅ straight or branched chain alcohols or mixtures thereof viz., methanol, ethanol, n-propanol, 2-propanol, n-butanol, tertiary butanol and n-pentanol or haloalkane solvents viz., chloroform, dichloromethane and dichloroethane or aromatic hydrocarbon solvents viz., benzene, toluene and exylene or cyclic ether solvents viz., tetrahydrofuran and dioxane or polar organic solvents viz., dimethyl formamide dimethyl sulfoxides, preferably a mixture of chloroform and dimethylsulfoxide wherein the ratio of (1) to chloroform is 1:3-10 w/v and the ratio of (I) to dimethyl sulfoxide is 1:1-3 w/v, in presence of oxidizing agents such as hydrogen peroxide, peracetic acid, 3-chloroperbenzoic acid, sodium hypo chlorite or sodium hypobromite, preferably 3-chloroperbenzoic acid wherein the ratio of (I) to 3-chloroperbenzoic acid is 1:0.5-1.5 w/w which is dissolved in one of the solvents recited above in chloroform. The temperature at which the oxidation can carried out can vary from −40° C. to boiling point of the solvent used, preferably 10 to 15° C. till the reaction is substantially complete.

Thereafter 10-15% w/v aqueous alkaline solution hydroxide, potassium hydroxide, sodium carbonate and potassium carbonate, preferably aqueous solution of sodium hydroxide is added to the reaction mixture. The pH of the reaction mixture is adjusted to 9-12 using an acid such as acetic acid, hydrochloric acid or hydrobromic acid. The resultant biphasic system thus obtained is separated and the organic layer is extracted with 1-5% w/v aqueous alkaline solution recited above, preferably aqueous solution of sodium hydroxide. This alkaline extract is diluted with a mixture of halo alkane solvent as recited above, preferably chloroform wherein the ratio of (1) to chloroform is 1:1-3 w/v and an alcohol or a mixture of alcohols as recited above, preferably methanol wherein the ratio of (I) to methanol is 1:1-3 w/v.

Then the pH of the mass is again adjusted to 9-12 with an acid as recited above, and the organic layer separated. To the separated organic layer is now added an ether solvent having C₁-C₄ straight or branched chain carbon atoms viz., dietheyl ether, methyl ethyl ether, diisobutyl ether, ditertiary butyl ether or tertiary butyl methyl wherein the ratio of (I) to ether is 1:3-7 w/v. The reaction mixture is then stirred till the complete crystallization, at a temperature of 0-25° C., preferably at 0-5° C. and subjected to filtration.

The resultant solid is then dissolved in a mixture of 10 to 20% w/v aqueous alkalkine solution as recited above, preferably aqueous solution of sodium hydroxide and an alcohol or a mixture of alcohols as recited above, preferably methanol. The pH is adjusted to 9-10, with an acid as described above, at 0-25° C., preferably 0-5° C. until complete crystallization. The 2-[[[4-(3-methoxypropoxy)-3-methyl-2-pyridine]-methyl]sulfinyl]-1H-benzimidazole thus obtained is filtered and dried.

The crystalline forms of Rabeprazole sodium of the present invention are also high melting solids with residual solvents within permissible limits and are very well suited for formulation.

The present invention also envisages pharmaceutical compositions prepared using Form X or Form Y of Rabreprazole (2-[[[4-(3-methoxypropoxy)-3-methyl-2-pyridinyl]-methyl]sulfinyl]-1H-benzimidazole sodium) and a physiologically acceptable carrier, diluent, excipient, additive, filler, lubricant, binder, stabilizer, solvent or solvate.

The pharmaceutical composition may be in a form normally employed, such as tablets, capsules, lozenges, powders, syrups, solutions, suspensions, ointments, dragees and the like, may contain flavourants, sweetners, etc. in suitable solid or liquid carriers or diluents, or in suitable sterile media to form injectable solutions or suspensions. Such compositions typically contain from 1 to 25%, preferably 1 to 15% by weight of active ingredient, the remainder of the composition being one or more of a pharmaceutically acceptable carrier, diluent, excipient, additive, filler, lubricant, binder, stabilizer, solvent or solvate.

The Form X or Form Y of Rabreprazole (2-[[[4-(3-methoxypropoxy)-3-methyl-2-pyridinyl]-methyl]sulfinyl]-1H-benzimidazole sodium can be administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred.

Dosage is in the range or about 0.01 to about 100 mg/kg body weight of the subject per day or preferably about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the compound can be combined with a suitable solid or liquid carrier or diluent to form capsules, tablets, powders, syrups, solutions, suspensions and the like. The pharmaceutical compositions, may, if desired, contain additional components such a flavourants, sweeteners, excipients and the like. For parenteral administration, the compound can be combined with sterile aqueous or organic media to form injectable solutions or suspensions. For example, solutions in sesame or peanut oil, aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the compound of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed. An effective amount means that amount of a drug or pharmaceutical agent that will elicit the biological or medical response of a tissue, system, human or animal sought.

The compositions may be prepared by methods known to those in the pharmaceutical field.

Form X and Form Y of Rabeprazole sodium show better chemical stability such as thermo stability and light stability as compared to prazoles such as panto prazole sodium and omeprazole sodium.

FIG. 1 is characteristic X-ray powder diffraction pattern of Form X of 2-[[[4-(3-methoxypropoxy)-3-methyl-2-pyridinyl]-methyl]sulfinyl]-1H-benzimidazole sodium (Rabeprazole sodium).

Vertical axis: Intensity (CPS); Horizontal axis: 20 values (in degrees) obtained are 5.13, 6.606, 7.244, 8.569, 9.353, 10.565, 12.161, 12.923, 14.414, 14.864, 16.372, 17.309, 18.173, 19.072, 20.01, 20.539, 22.177, 23.469, 24.81 and 25.494.

FIG. 2 is Differential Scanning Calorimetry thermogram of Form X of 2-[[[4-(3-methoxypropoxy)-3-methyl-2-pyridinyl]-methyl]sulfinyl]-1H-benzimidazole sodium (Rabeprazole sodium). The Differential Scanning Calorimetry thermogram exhibits a significant endo-exo pattern at 154.62° C. and 214.65° C. The heating rate for the DSC is 5 deg./minute.

FIG. 3 is characteristic X-ray powder diffraction pattern of Form Y of 2-[[[4-(3-methoxypropoxy)-3-methyl-2-pyridinyl]-methyl]sulfinyl]-1H-benzimidazole sodium (Rabeprazole sodium).

Vertical axis: Intensity (CPS); Horizontal axis: 20 (degrees). The significant 2θ values (in degrees) obtained are 5.61, 7.207, 7.725, 9.649, 10.352, 11.231, 14.546, 16.418, 16.899, 18.816, 19.442 and 24.943.

FIG. 4 is Differential Scanning Colorometry thermogram of Form Y of 2-[[[4-(3-methoxypropoxy)-3-methyl-2-pyridinyl]-methyl]sulfinyl]-1H-benzimidazole sodium (Rabeprazole sodium). The Differential Scanning Calorimetry thermogram exhibits a significant endo-exo pattern at 182.61° C. and 215.57° C.

The heating rate for the DSC is 5 deg./minute.

The present invention is illustrated by the following examples, which are not intended to limit the effective scope of the claims.

### REFERENCE EXAMPLE

2-[{4-(3-methoxypropoxy)-3-methylpyridine-2-yl}methylthio]-1H-benzimidazole (prepared as per example 90 of the U.S. Pat. No. 5,045,552) (100 grams, 0.29 moles) is added to a mixture of chloroform (500 ml) and dimethylsulfoxide (200 ml) and the reaction mixture is cooled to −10 to −15° C. 3-chloroperbenzoic acid (60 grams, 0.24 moles) is dissolved in chloroform (500 ml), and added to the above solution at −10 to −15° C. for about 1-12 hours and the reaction mixtures maintained at the same temperature for 30 minutes. Thereafter 12.8% w/v aqueous sodium hydroxide solution (500 ml) is added to the reaction mixture. The pH of the reaction mixture is adjusted to 9.5 to 10.0 with acetic acid. Of the biphasic system thus obtained the organic layer is separated and then extracted with 1.6% w/v aqueous sodium hydroxide solution (500 ml). Further the sodium hydroxide extract is diluted with a mixture of chloroform (140 ml) and methanol (100 ml). Then the pH of the mass is again adjusted to 9.5 to 10.0 with acetic acid and the organic layer separated again. To the separated organic layer is now added tert.butyl methyl ether (440 ml). The reaction mixture is stirred for about 1-12 hours at a temperature of 0-5° C. and subjected to filtration. The residue is dissolved in a mixture of 10% w/v aqueous sodium hydroxide solution (100 ml) and methanol (65 ml). The pH is adjusted to 9.0 to 9.5 with acetic acid at 10-15° C. and further stirred for 12 hours followed by filtration. The wet material is then dissolved in dichloromethane (130 ml) and the water layer separated where after the solution is added to tert.butyl methyl ether (260 ml), stirred at a temperature of 0-5° C. for 1-2 hours. The 2-[[[4-(3-methoxypropoxy)-3-methyl-2-pyridinyl]-methyl]sulfinyl]-1H-benzimidazole thus obtained is filtered and dried.

### Example 1

### Preparation of Crystalline Form-X of Rabeprazole Sodium

2-[[[4-(3-methoxypropoxy)-3-methyl-2-pyridinyl]-methyl]sulfinyl]-1H-benzimidazole (obtained as per reference example) (50.0 grams, 0.139 moles) is dissolved in a mixture of sodium hydroxide (7.5 grams, 0.187 moles) and methanol (100.0 ml) and stirred at ambient temperature. The reaction solution is filtered through hi-flow and washed with methanol (50.0 ml). Methanol from the filtrate is distilled off under high vacuum. The reaction mass is cooled to ambient temperature followed by addition of dichloromethane (100.0 ml) accompanied by distillation to remove traces of methanol. Dichloromethane (50.0 ml) and petroleum ether (100.0 ml) is then added to the residual mass, which is then stirred at 25-30° C. for about 6-8 hours. The solid that is obtained further diluted with petroleum ether (150 ml) and stirred at 25-30° C. for about 6-8 hours. The precipitated solid is filtered and washed with petroleum ether (100.0 ml) and dried at 50-60° C. for 12 hours to afford the desired Form X of Rabeprazole sodium (Weight: 50.4 grams, 94.9%).

The X-ray Diffraction Pattern, Differential Scanning Calorimetry thermogram of Form X of Rabeprazole sodium obtained in above example is in accordance with FIGS. 1 and 2 respectively.

### EXAMPLE 2

### Preparation of Crystalline Form-Y of Rabenrazole Sodium

2-[[[4-(3-methoxypropoxy)-3-methyl-2-pyridinyl]-methyl]sulfinyl]-1H-benzimidazole (obtained as per reference example) (750.0 grams, 2.089 moles) is dissolved in a mixture of sodium hydroxide (112.5 grams, 2.8125 moles) and methanol (1500.0 ml) and stirred at ambient temperature. The reaction solution is filtered through hi-flow and washed with methanol (750.0 ml). Methanol from the filtrate is distilled off under high vacuum. The reaction mass is cooled to ambient temperature followed by addition of dichloromethane (1500.0 ml) accompanied by distillation to remove traces of methanol. The reaction mass is cooled to ambient temperature and n-butanol (375.0 ml) and tertiary butyl methyl ether (6.0 lit) is added to the residual mass which is stirred at 25-30° C. for 6-8 hours. The reaction mixture is further cooled to 5-15° C. and then stirred for another 3-5 hours. The solid is thus obtained is filtered and washed with tertiary butyl methyl ether (1500.0 ml) and dried at 50-60° C. for 7 hours to afford the desired crystalline Form Y of Rabeprazole sodium (Weight: 725.0 grams 91.1%).

The X-ray Diffraction Pattern, Differential Scanning Calorimetry thermogram of Form Y of Rabeprazole sodium obtained in the example is in accordance with FIGS. 3 and 4 respectively.

1. A crystalline Form X of 2-[[[4-(3-methoxypropoxy)-3-methyl-2 pyridinyl]-methyl]sulfinyl]-1H-benzimidazole and hydrates thereof.

2. The crystalline Form X of Rabeprazole sodium of claim 1 having an X-ray powder diffraction pattern with peaks at about 5.13, 6.606, 7.244, 8.569, 9.353, 10.565, 12.161, 12.923, 14.414, 14.864, 16.372, 17.309, 18.173, 19.072, 20.01, 20.539, 22.177, 23.469, 24.81 and 25.494 (degrees 2 theta).

3. The crystalline Form X of Rabeprazole sodium of claim 1 having an X-ray powder diffraction pattern substantially as depicted in FIG. 1.

4. The crystalline Form X of Rabeprazole sodium of claim 1 having a differential scanning calorimetery thermogram which exhibits a significant endo-exo pattern at about 154.62° C. and about 214.65° C.

5. The crystalline Form X of Rabeprazole sodium of claim 1 having a Differential Scanning Calorimetry thermogram substantially as depicted in FIG. 2.

6. The crystalline Form X of Rabeprazole sodium of claim 1 having a melting range of 140-150° C.

7. A process for preparing Form X of 2-[[[4-(3-methoxypropoxy)-3-methyl 2-pyridinyl]-methyl]sulfinyl]-1H-benzimidazole sodium, which comprises:
a) dissolving 2-[[[4-(3-methoxypropoxy)-3-methyl-2-pyridinyl]methyl]sulfinyl]-1H-benzimidazole in a C₁-C₄ alkanol of sodium hydroxide or mixtures thereof, and distilling the solvent from the reaction solution;
b) adding a chlorinated C₁-C₃ hydrocarbon solvent to a residual mass obtained in step a);
c) distilling the chlorinated solvent azeotropically under reduced pressure from a material of b);
d) adding a chlorinated C₁-C₃ hydrocarbon solvent and a C₅-C₁₀ alkane solvent or C₅-C₁₀ cyclic alkane; or mixtures thereof; accompanied by stirring, and
e) isolating Form X of 2-[[[4-(3-methoxypropoxy)-3-methyl-2 pyridinyl]-methyl]-sulfinyl]-1H-benzimidazole sodium.

8. A crystalline Form Y of 2-[[[4-(3-methoxypropoxy)-3-methyl-2 pyridinyl]-methyl]sulfinyl]-1H-benzimidazole sodium and hydrates thereof.

9. The crystalline Form Y of Rabeprazole sodium of claim 8, having an X-ray powder diffraction pattern with peaks at about 5.61, 7.207, 7.725, 9.649, 10.352, 11.231, 14.546, 16.418, 16.899, 18.816, 19.442 and 24.943 (degrees 2 theta).

10. The crystalline Form Y of Rabeprazole sodium of claim 8, having an X-ray powder diffraction pattern substantially as depicted in FIG. 3.

11. The crystalline Form Y of Rabeprazole sodium of claim 8, having a differential scanning calorimetery thermogram which exhibits a significant endo-exo pattern at about 182.61° C. and about 215.57° C.

12. The crystalline Form Y of Rabeprazole sodium of claim 8, having a Differential Scanning Calorimetry thermogram substantially as depicted in FIG. 4.

13. The crystalline Form Y of rabeirazole sodium of claim 7, having a melting range of 160-170° C.

14. A process for preparing Form Y of 2-[[[4-(3-methoxypropoxy)-3-methyl-2-pyridinyl]-methyl]sulfinyl]-1H-benzimidazole sodium, which comprises:
a) dissolving 2-[[[4-(3-methoxypropoxy)-3-methyl-2-pyridinyl]-methyl]sulfinyl]-1H-benzimidazole in a C₁-C₄ alkanol of sodium hydroxide, and distilling the solvent from the solution;
b) optionally adding a chlorinated C₁-C₃ hydrocarbon solvent to a residual mass obtained in step a);
c) distilling the chlorinated solvent of azeotropically under reduced pressure from a material of b);
d) adding to a residue obtained in step c) either a C₃-C₅ straight or branched chain alcohol and an ether solvent, accompanied by stirring; and
e) isolating Form Y of 2-[[[4-(3-methoxypropoxy)-3-methyl-2-pyridinyl]-methyl]sulfinyl]-1H-benzimidazole sodium.

15. A composition comprising a crystalline Form X of 2-[[[4-(3-methoxypropoxy)-3-methyl-2-pyridinyl]-methyl]sulfinyl]-1H-benzimidazole sodium or hydrates thereof according to claim 1 and at least one of a physiologically acceptable carrier, diluent, excipient, additive, filler, lubricant, binder, stabilizer, solvent or solvate.

16. A composition comprising a crystalline Form Y of 2-[[[4-(3-methoxypropoxy)-3-methyl-2-pyridinyl]-methyl]sulfinyl]-1H-benzimidazole sodium or hydrates thereof according to claim 8 and at least one of a physiologically acceptable carrier, diluent, excipient, additive, filler, lubricant, binder, stabilizer, solvent or solvate.
